(19) United States
(12) Patent Application Publication
(10) Pub. No.: US 2012/0077978 A1
(43) Pub. Date: Mar. 29, 2012

(54) Structure Based Design Of Inhibitors Of Human Thymidylate Synthase

(75) Inventors: Lukasz Lebioda, Columbia, SC (US); Lesa R. Celeste, Irmo, SC (US); Leslie L. Lovelace, Orangeburg, SC (US)

(73) Assignee: UNIVERSITY OF SOUTH CAROLINA, Columbia, SC (US)

(21) Appl. No.: 13/247,303

(22) Filed: Sep. 28, 2011

Related U.S. Application Data
(60) Provisional application No. 61/387,053, filed on Sep. 28, 2010.

(51) Int. Cl. C07F 9/06; C07F 9/38; C07F 9/28; C07D 487/04

(52) U.S. Cl. 544/244; 544/250

(57) ABSTRACT

A compound is provided having the chemical structure:

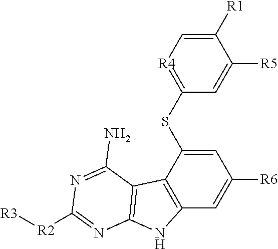

where R1 is H, CH₃, a tert-butyl group, C(═O)-Glu, C(═O)-Asp, C(═O)-Gln, C(═O)-Asn, or R₅ is C(═O)-Glu, C(═O)-Asp, C(═O)-Gln, or C(═O)-Asn; R2 comprises an organic chain that is 1 to 5 atoms in length; R3 is a tetrahedral moiety having a central atom and three terminal atoms, wherein the central atom is P, S, V, W, Se, or Te and wherein the terminal atoms are O and/or F; R4 is CH, N, or S; R5 is H, CH₃; CH₂C₆H₆, CH₂CH₂C₆H₆, OCH₂C₆H₆, C(═O)-Phe, CH₂C(═O)-Phe, C(═O)-Tyr, CH₂C(═O)-Tyr, or a tert-butyl group; and R6 is H, OH, CH₃, CH₂OH, or a tert-butyl group.

### PRIORITY INFORMATION

The present application claims priority to U.S. Provisional Patent Application Ser. No. 61/387,053 filed on Sep. 28, 2010 titled “Structure Based Design of Inhibitors of Human Thymidylate Synthase” of Lebioda, et al., the disclosure of which is incorporated by reference herein.

### GOVERNMENT SUPPORT CLAUSE

This invention was made with government support under contract number CA 076560 awarded by the National Institutes of Health (NIH). The government has certain rights in the invention.

### BACKGROUND

Colorectal cancer is the second most frequent cause of cancer mortality with approximately 50,000 deaths annually in the U.S (1). Fluoropyrimidine (FP) agents, such as 5-fluorouracil (FUra), 5-fluorodeoxyuridine (FdUrd), and the recently approved FUra pro-drug, capecitabine, represent an important class of antimetabolites utilized in the therapy of carcimonas of the gastrointestinal tract (4). An important mode of action of FPs is via the metabolite, 5-fluorodeoxyuridylate-monophosphate (FdUMP), which is a potent inhibitor of the enzyme, thymidylate synthase (TS). Thymidylate synthase (TS) is an enzyme that is the sole source of thymidylate, a building block of DNA. TS inhibition leads to the death of rapidly dividing cells, such as cancer cells. Therefore, human TS (hTS) is the target of chemotherapeutic agents, which bind at the active site of the enzyme, including pro-drugs of FdUMP, antifolates: pemetrexed (Alimta®, LY-231514, Eli Lilly) and raltitrexed (Tomudex®, ZD1694, Astra Zeneca) and other drugs currently in development. In patients with advanced cancers of the gastrointestinal tract, the effectiveness of existing therapy is poor, 30%, primarily as a result of emerging resistance. The resistance is often associated with increased levels of hTS in cancer cells.

TS catalyzes the reductive methylation of deoxyuridylate (dUMP) to form thymidylate (dTMP); the source of the transferred methyl group is the enzyme co-substrate, 5,10-methylenetetrahydrofolate (CH₂H₄PteGlu). Structural analogs of CH₂H₄PteGlu, including pemetrexed (Alimta®), raltitrexed (Tomudex®, ZD1694) and several agents in current development are a second class of TS inhibitors (5,6). Since TS provides the sole de novo source of dTMP, the enzyme is essential for DNA synthesis and cell growth. Inhibition of TS is cytotoxic for rapidly dividing cells and leads to so called “thymineless death”. The chief obstacles to successful therapeutic intervention in the therapy of colorectal cancer are metastases, particularly to the liver, and drug resistance. In patients whose cancer has spread to distant sites such as the liver, the prognosis is grim. Approximately 30% of patients with disseminated cancer respond to first-line therapy (3). The poor response rate to chemotherapeutic drugs is thought to be due to innate and acquired resistance. Development of new TS inhibitors should provide new therapeutic agents needed to avoid resistance, likely through a multi-drug approaches.

Basically there are two classes of TS inhibitors; they are either nucleotide analogues such as FdUMP or antifolates that bind to TS-dUMP complex. Dr. Aleem Gangjee at Duquesne University has developed TS inhibitors that do not belong to either category. These TS inhibitors are shown as the Prior Art Compounds below (where R₁ is H or CH₃):

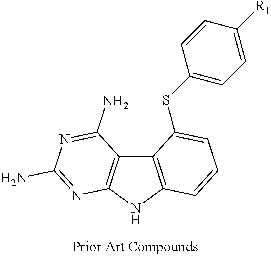

These TS inhibitors have been shown to have IC₅₀ concentrations similar to those of currently used chemotherapy drugs. However, the toxic effects of such drugs dictate the need for reduced dose concentrations.

As such, a need exists for next generation TS inhibitors with improved inhibitory properties and reduced IC₅₀ concentrations. Modifying the Prior Art Compounds to improve inhibition by reducing the IC₅₀ concentrations should lead to a more effective TS inhibitor.

### SUMMARY

Objects and advantages of the invention will be set forth in part in the following description, or may be obvious from the description, or may be learned through practice of the invention.

A compound is generally provided having the chemical structure:

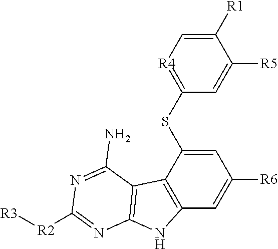

where R1 is H, CH₃, a tert-butyl group, C(═O)-Glu, C(═O)-Asp, C(═O)-Gln, C(═O)-Asn, or R₅ is C(═O)-Glu, C(═O)-Asp, C(═O)-Gln, or C(═O)-Asn; R2 comprises an organic chain that is 1 to 5 atoms in length; R3 is a tetrahedral moiety having a central atom and three terminal atoms, wherein the central atom is P, S, V, W, Se, or Te and wherein the terminal atoms are O and/or F; R4 is CH, N, or S; R5 is H, CH₃; CH₂C₆H₆, CH₂CH₂C₆H₆, OCH₂C₆H₆, C(═O)-Phe, CH₂C(═O)-Phe, C(═O)-Tyr, CH₂C(═O)-Tyr, or a tert-butyl group; and R6 is H, OH, CH₃, CH₂OH, or a tert-butyl group.

Methods of making the disclosed compounds are also provided.

Other features and aspects of the present invention are discussed in greater detail below.

### BRIEF DESCRIPTION OF THE DRAWINGS

A full and enabling disclosure of the present invention, including the best mode thereof to one skilled in the art, is set forth more particularly in the remainder of the specification, which includes reference to the accompanying figures, in which:

FIG. 1 shows the interaction of hTS with the Prior Art Compound; and

FIG. 2 shows the hTS active site complexed with either Compound 3 or Compound 4 as the inhibitor of hTS.

### DETAILED DESCRIPTION

Reference now will be made to the embodiments of the invention, one or more examples of which are set forth below. Each example is provided by way of an explanation of the invention, not as a limitation of the invention. In fact, it will be apparent to those skilled in the art that various modifications and variations can be made in the invention without departing from the scope or spirit of the invention. For instance, features illustrated or described as one embodiment can be used on another embodiment to yield still a further embodiment. Thus, it is intended that the present invention cover such modifications and variations as come within the scope of the appended claims and their equivalents. It is to be understood by one of ordinary skill in the art that the present discussion is a description of exemplary embodiments only, and is not intended as limiting the broader aspects of the present invention, which broader aspects are embodied exemplary constructions.

Chemical elements are discussed in the present disclosure using their common chemical abbreviation, such as commonly found on a periodic table of elements. For example, hydrogen is represented by its common chemical abbreviation H; helium is represented by its common chemical abbreviation He; and so forth.

The term “organic” is used herein to refer to a class of chemical compounds that are comprised of carbon atoms. For example, an “organic chain” is a chain of atoms that are covalently bonded together and includes carbon atoms in the main chain, but may also include other atoms either in the main chain and/or in side chains extending from the chain (e.g., oxygen, nitrogen, sulfur, etc.).

Generally speaking, the present disclosure is directed toward TS inhibitors shown as Compound 1. Compound 1 generally includes a tetrahedral moiety having three terminal atoms (e.g., any combination of oxygen and/or fluorine atoms) connected to a central atom (P, S, or other central atoms such as V, W, Se, Te, etc):

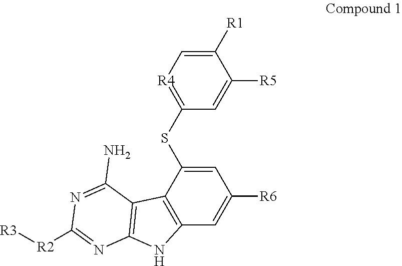

where R1 is H, CH₃, a tert-butyl group, C(═O)-Glu, C(═O)-Asp, C(═O)-Gln, C(═O)-Asn, or R₅ is C(═O)-Glu, C(═O)-Asp, C(═O)-Gln, or C(═O)-Asn; R2 comprises an organic chain that is 1 to 5 atoms in length; R3 is a tetrahedral moiety having a central atom and three terminal atoms, wherein the central atom is P, S, V, W, Se, or Te and wherein the terminal atoms are O and/or F; R4 is CH, N, or S; R5 is H, CH₃; CH₂C₆H₆, CH₂CH₂C₆H₆, OCH₂C₆H₆, C(═O)-Phe, CH₂C(═O)-Phe, C(═O)-Tyr, CH₂C(═O)-Tyr, or a tert-butyl group; and R6 is H, OH, CH₃, CH₂OH, or a tert-butyl group.

As shown, the tetrahedral moiety (R3) is positioned on a side chain (R2) of Compound 1. Particularly suitable tetrahedral moieties include those where the central atom is P or S, including by not limited to a phosphonate terminal group, a phosphate terminal group, a sulfonate terminal group, or a sulfate terminal group. However, one, two, or all of the oxygen atoms can be substituted with fluorine atoms in such tetrahedral moiety. Although this side chain is described as a tetrahedral moiety (i.e., a terminal-tetrahedron), it is noted that the angles may or may not be symmetric. As such, the terminal oxygen and/or fluorine atoms may not form a perfect tetrahedron.

Compound 1 can yield better inhibition of TS than that of the Prior Art Compounds through the tetrahedral moiety (e.g., —R₂-R₃—O₃ or —R₂-R₃—F₃), which can strongly increase binding to TS enabling increased inhibition.

As stated, R₂ can be an organic chain that is 1 to 5 atoms in length, such as 2 to 4 atoms in length. In one particular embodiment, R₂ can be 3 atoms in length. As used herein, the term “organic chain” refers to a hydrocarbon that forms a covalent link from the terminal tetrahedral moiety group to the aromatic ring (e.g., the modified purine ring).

The organic chain of R2 generally includes at least one carbon atom, and may be a saturated hydrocarbon (e.g., an alkyl chain), an unsaturated hydrocarbon (e.g., an alkene chain), or may include other atoms (e.g., oxygen, nitrogen, sulfur, etc.), such as an ether chain, an ester chain, etc. In one particular embodiment, R₂ can be an alkane chain that is 1 to 5 carbon atoms in length, such as 2 to 4 carbon atoms in length (e.g., 3 carbon atoms in length). In an alternative embodiment, the R₂ can be an ether chain (i.e., including an oxygen atom in the chain). For example, the ether chain can have 1 to 4 carbon atoms and 1 oxygen atom, such as 2 to 3 carbon atoms and 1 oxygen atom (e.g., 2 carbon atoms and 1 oxygen atom). In one particular embodiment, the oxygen atom can be positioned adjacent to the terminal tetrahedral moiety (e.g., forming a phosphate or sulfate terminal group).

Other modifications than those R groups shown may also be made using Compound 1 as a base molecule, assuming that the general function of the compound is not substantially disturbed. Other possibilities of side group modifications include R₁ being a tert-butyl group or R₅ being a tert-butyl group or R₆ being a tert-butyl group. Additionally, a carbonyl could be present in a side chain followed by a polar uncharged or polar acidic residue, where R₁ is C(═O)-Glu, C(═O)-Asp, C(═O)-Gln, C(═O)-Asn, or R₅ is C(═O)-Glu, C(═O)-Asp, C(═O)-Gln, or C(═O)-Asn.

In particular embodiments, R1 can be H or CH₃, R4 can be CH, R5 can be H, and/or R6 can be H. For example, Compound 2 is shown below as one particular embodiment encompassed by Compound 1 above, where R3 defines a phosphonate moiety (i.e., the central atom is P and all three terminal atoms are O), R5 is H, and R6 is H:

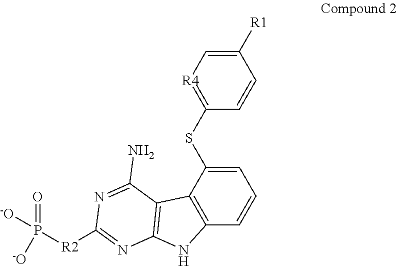

where R₂ is as discussed above with respect to Compound 1 (i.e., comprises an organic chain that is 1 to 5 atoms in length); R₁ is CH₃ or H; R₄ is CH, N or S.

Two particularly suitable TS inhibitors are shown in Compound 3 and Compound 4, having a phosphonate and phosphate moiety side chains, respectively, extending 3 atoms from the aromatic ring (i.e., from the 2 position of the modified purine ring):

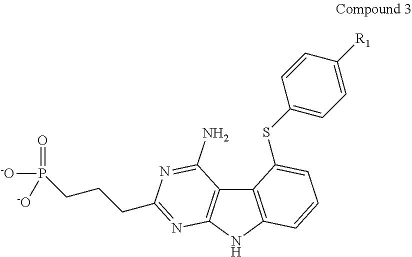

where R₁ is H or CH₃; and

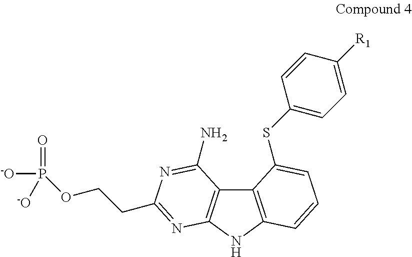

where R₁ is H or CH₃.

Compound 5 shows other embodiments of Compound 1 where R1 can be H or CH₃, R3 defines a sulfonate moiety (i.e., the central atom is S and all three terminal atoms are O), R4 is CH, R5 is H, and R6 is H:

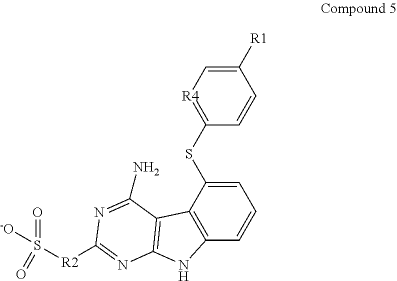

where R1 is H or CH₃; R2 comprises an organic chain that is 1 to 5 atoms in length; and R4 is CH, N, or S. For example, the organic chain of R2 can be 3 atoms in length, and wherein at least 2 of the atoms are carbon atoms, such as shown in the embodiments of Compounds 6 and 7:

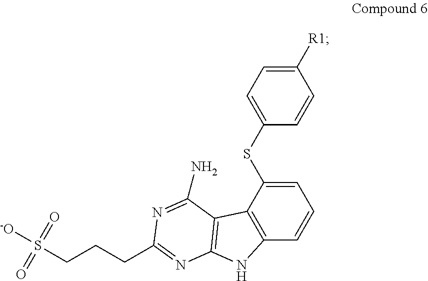

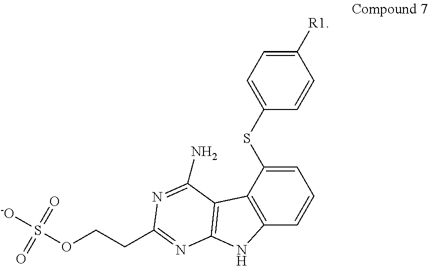

Embodiments where all three terminal atoms are F are also provided, such as shown in Compound 8:

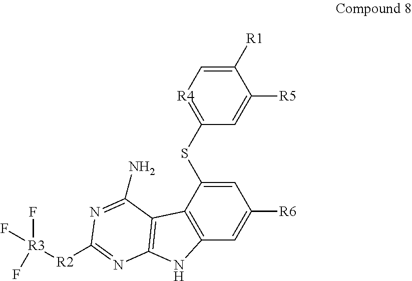

where R1 is H, CH₃, a tert-butyl group, C(═O)-Glu, C(═O)-Asp, C(═O)-Gln, C(═O)-Asn, or R₅ is C(═O)-Glu, C(═O)-Asp, C(═O)-Gln, or C(═O)-Asn; R2 comprises an organic chain that is 1 to 5 atoms in length; R3 is P, S, V, W, Se, or Te; R4 is CH, N, or S; R5 is H, CH₃; CH₂C₆H₆, CH₂CH₂C₆H₆, OCH₂C₆H₆, C(═O)-Phe, CH₂C(═O)-Phe, C(═O)-Tyr, CH₂C(═O)-Tyr, or a tert-butyl group; and R6 is H, OH, CH₃, CH₂OH, or a tert-butyl group.

Compound 9 shows one particular embodiment encompassed by Compound 8:

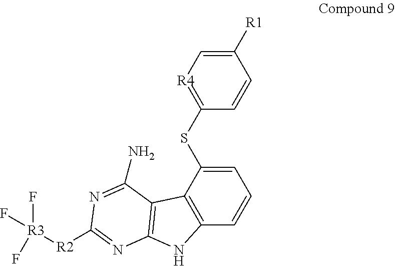

where R1 is H or CH₃; R2 comprises an organic chain that is 1 to 5 atoms in length; R3 is P; and R4 is CH, N, or S. As stated above, the organic chain of R2 can be, in certain embodiments, 2 to 4 atoms in length. For example, the organic chain of R2 can be 3 atoms in length, and wherein at least 2 of the atoms are carbon atoms, such as shown in the embodiments of Compounds 10 and 11:

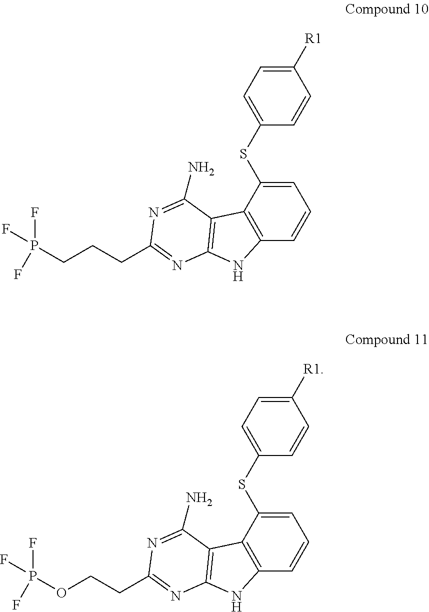

In the embodiments shown in Compounds 10 and 11, the central atom is P and all three terminal atoms are F.

### EXAMPLES

A new crystal form of human TS (hTS) was developed to allow diffusion of drug candidates and monitoring their mode of interaction with the hTS target. This crystal form was developed using a mutant of hTS that shifts the equilibrium to the active conformation. Destabilization of the inactive conformation of hTS was achieved by replacing Arg163 with a smaller residue that was unable to form hydrogen bonds with the peptide carbonyls. This residue is not conserved in either bacterial TSs or rodent TSs, for which the inactive conformation has not been observed; in E. coli TS (ecTS) the residue at the 163 position is Thr and in rodents it is Lys. We have generated these mutants and have demonstrated that R163K has a higher catalytic activity than wild-type hTS. In addition, this mutant is not subject to inhibition by phosphate ions as is the wild-type enzyme.

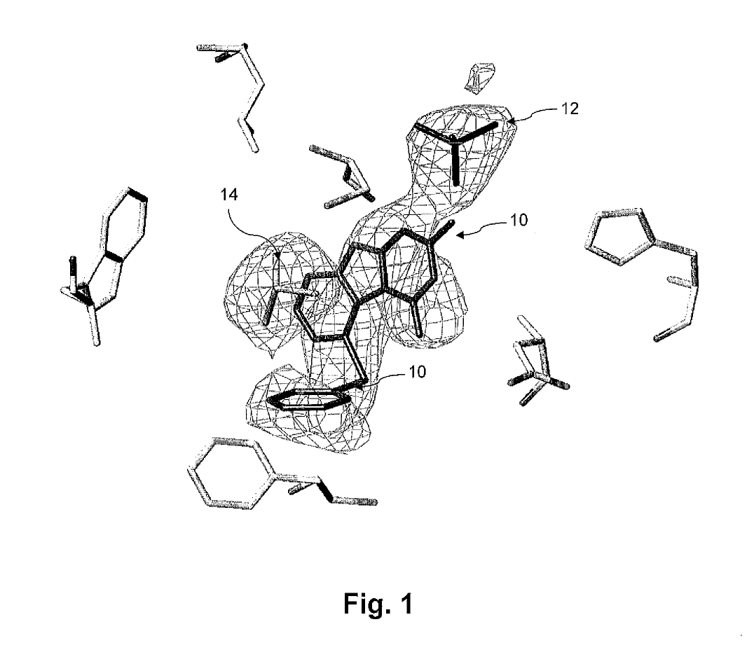

Subsequently, X-ray crystallography at 3.0 Å resolution was used to determine the three-dimensional structure of a complex between hTS and the Prior Art Compound (where R₁ is H) (provided by Dr. Aleem Gangjee, Graduate School of Pharmaceutical Sciences, Duquesne University). The interaction of hTS with Prior Art Compound is shown in FIG. 1. Specifically, FIG. 1 shows the hTS active site complexed with the Prior Art Compound (where R₁ is H) with electron density observed in the Fₒ-F_{c} map. As shown, a phosphate ion is present within the active site. hTS is shown with the Prior Art Compound 10, a phosphate ion 12, and a DMSO molecule 14.

From these x-ray crystallographic studies of hTS with Prior Art Compound, it was apparent that the mode of binding of the Prior Art Compound is different from the modes in which all other known inhibitors of hTS bind. Furthermore, it does not belong in either of the known classes of TS inhibitors (i.e., nucleotide analogues such as FdUMP or antifolates that bind to the TS-dUMP complex).

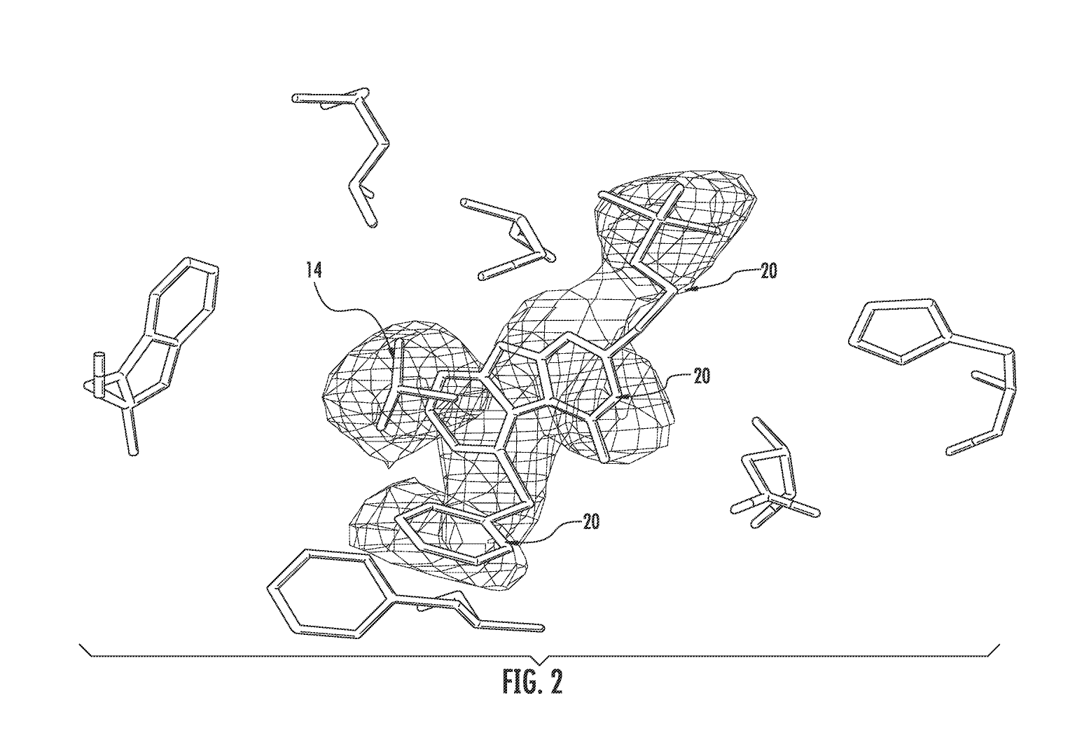

Based on this structure, modifications to the Prior Art Compound were theorized that should improve their inhibitory properties. Specifically, Compound 3 and Compound 4 discussed above were modeled into the active site of hTS using the software package TURBO-FRODO. The modeling of this interaction is shown in FIG. 2. Specifically, FIG. 2 shows the hTS active site complexed with either Compound 3 or Compound 4 as the inhibitor of hTS. hTS is shown with Compound 3 or Compound 4 (20) and a DMSO molecule 22.

These and other modifications and variations to the present invention may be practiced by those of ordinary skill in the art, without departing from the spirit and scope of the present invention, which is more particularly set forth in the appended claims. In addition, it should be understood the aspects of the various embodiments may be interchanged both in whole or in part. Furthermore, those of ordinary skill in the art will appreciate that the foregoing description is by way of example only, and is not intended to limit the invention so further described in the appended claims.

What is claimed:

1. A compound having the chemical structure: 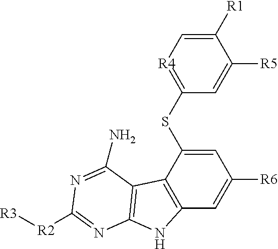
where
R1 is H, CH₃, a tert-butyl group, C(═O)-Glu, C(═O)-Asp, C(═O)-Gln, C(═O)-Asn, or R₅ is C(═O)-Glu, C(═O)-Asp, C(═O)-Gln, or C(═O)-Asn;
R2 comprises an organic chain that is 1 to 5 atoms in length;
R3 is a tetrahedral moiety having a central atom and three terminal atoms, wherein the central atom is P, S, V, W, Se, or Te and wherein the terminal atoms are O and/or F;
R4 is CH, N, or S;
R5 is H, CH₃; CH₂C₆H₆, CH₂CH₂C₆H₆, OCH₂C₆H₆, C(═O)-Phe, CH₂C(═O)-Phe, C(═O)-Tyr, CH₂C(═O)-Tyr, or a tert-butyl group; and
R6 is H, OH, CH₃, CH₂OH, or a tert-butyl group.

2. The compound of claim 1, wherein the organic chain of R2 is 2 to 4 atoms in length.

3. The compound of claim 1, wherein the organic chain of R2 comprises an alkyl chain.

4. The compound of claim 1, wherein the organic chain of R2 comprises an ether chain.

5. The compound of claim 1, wherein the organic chain of R2 is 3 atoms in length, wherein at least 2 of the atoms are carbon atoms.

6. The compound of claim 5, wherein the organic chain comprises 3 carbon atoms.

7. The compound of claim 5, wherein the organic chain comprises an oxygen atom and two carbon atoms.

8. The compound of claim 1, wherein R1 is H or CH₃.

9. The compound of claim 1, wherein the central atom in R3 is P or S.

10. The compound of claim 1, wherein R4 is CH.

11. The compound of claim 1, wherein R5 is H.

12. The compound of claim 1, wherein R6 is H.

13. The compound of claim 1, having the chemical structure: 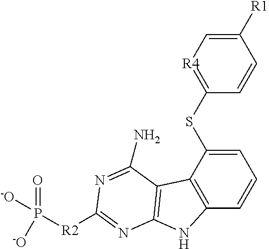
where
R1 is H or CH₃;
R2 comprises an organic chain that is 1 to 5 atoms in length; and
R4 is CH, N, or S.

14. The compound of claim 13, wherein the organic chain of R2 is 2 to 4 atoms in length.

15. The compound of claim 13, wherein the organic chain of R2 comprises an alkyl chain or an ether chain.

16. The compound of claim 13, wherein the organic chain of R2 is 3 atoms in length, and wherein at least 2 of the atoms are carbon atoms.

17. The compound of claim 16, having the chemical structure:

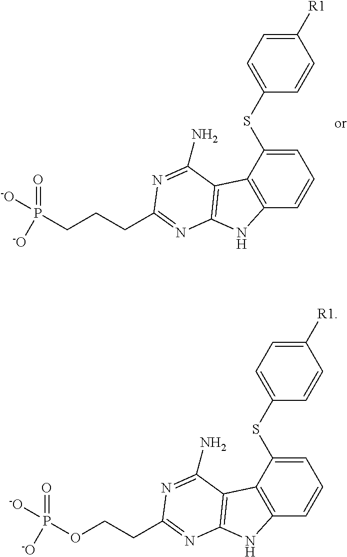

18. The compound of claim 1, having the chemical structure: 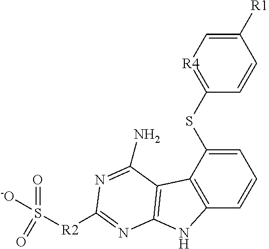
where
R1 is H or CH₃;
R2 comprises an organic chain that is 1 to 5 atoms in length; and
R4 is CH, N, or S.

19. The compound of claim 18, wherein the organic chain of R2 is 2 to 4 atoms in length.

20. The compound of claim 18, wherein the organic chain of R2 comprises an alkyl chain or an ether chain.

21. The compound of claim 18, wherein the organic chain of R2 is 3 atoms in length, and wherein at least 2 of the atoms are carbon atoms.

22. The compound of claim 21, having the chemical structure:

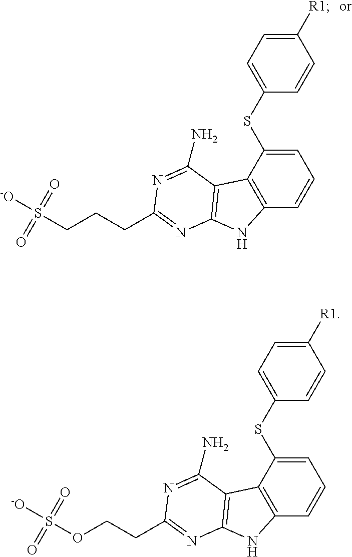

23. The compound of claim 1, having the chemical structure: 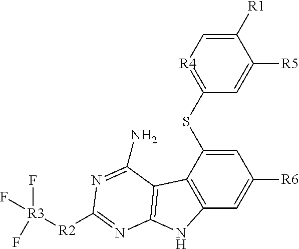
where
R1 is H, CH₃, a tert-butyl group, C(═O)-Glu, C(═O)-Asp, C(═O)-Gln, C(═O)-Asn, or R₅ is C(═O)-Glu, C(═O)-Asp, C(═O)-Gln, or C(═O)-Asn;
R2 comprises an organic chain that is 1 to 5 atoms in length;
R3 is P, S, V, W, Se, or Te;
R4 is CH, N, or S;
R5 is H, CH₃; CH₂C₆H₆, CH₂CH₂C₆H₆, OCH₂C₆H₆, C(═O)-Phe, CH₂C(═O)-Phe, C(═O)-Tyr, CH₂C(═O)-Tyr, or a tert-butyl group; and
R6 is H, OH, CH₃, CH₂OH, or a tert-butyl group.

24. The compound of claim 23, having the chemical structure: 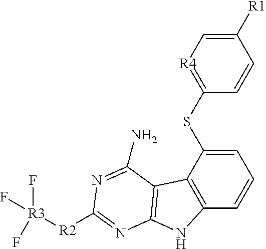
where
R1 is H or CH₃;
R2 comprises an organic chain that is 1 to 5 atoms in length;
R3 is P; and
R4 is CH, N, or S.

25. The compound of claim 24, wherein the organic chain of R2 is 2 to 4 atoms in length.

26. The compound of claim 25, wherein the organic chain of R2 is 3 atoms in length, and wherein at least 2 of the atoms are carbon atoms.

27. The compound of claim 26, having the chemical structure: [chemical structure]
